(19) United States
(12) Patent Application Publication
(10) Pub. No.: US 2018/0028206 A1
(43) Pub. Date: Feb. 1, 2018

(54) Multi-Lumen Catheter for Cardiac Device Deployment

(71) Applicant: VIMAL NANAVATI, Chula Vista, CA (US)

(72) Inventor: VIMAL NANAVATI, Chula Vista, CA (US)

(21) Appl. No.: 15/782,681

(22) Filed: Oct. 12, 2017

Related U.S. Application Data
(63) Continuation-in-part of application No. 14/684,152, filed on Apr. 10, 2015, now abandoned.

(51) Int. Cl. A61B 17/221; A61B 17/12

(52) U.S. Cl. CPC A61B 17/221; A61B 17/12122; A61B 2017/22038

(57) ABSTRACT

A implant device delivery system and method is provided employing a sheath with three lumens configured for translation of a shapeable guidewire and a balloon device and an implant such as a cardiac device. The balloon and balloon wire extend from an aperture spaced from the distal end of the sheath and form a rail upon anchoring of the balloon. An extension portion of the sheath is bendable to adjust an angle of deployment of the implant.

This Application is a Continuation in Part to U.S. patent application Ser. No. 14/684,152 filed on Apr. 10, 2015, which is incorporated herein in its entirety by this reference thereto.

The disclosed technology relates generally to catheters and, more specifically, to multi-lumen catheters for device deployment in the cardiovascular system.

### TECHNICAL FIELD

### BACKGROUND

Heart disease and related heart conditions continue to be a serious health risk to the public at large. For example, atrial fibrillation is a serious medical condition that occurs when the atrial chamber beats out of rhythmic coordination with the ventricle chambers of the heart. If continuously left untreated, atrial fibrillation may cause the heart to weaken or prevent the blood from pumping effectively, thus increasing the likelihood of a heart failure or stroke.

Effective treatment options may include sealing the left atrial appendage with a cardiac device to help reduce the formation of clots in the left atrial appendage and minimizing the likelihood of a stroke. A catheter system may be used to deploy devices throughout the vascular system. For example, a catheter system may be used to deploy a cardiac device to specific locations within the heart (e.g., the left atrium). Conventional catheter technology, however, does not allow for efficient manipulation of cavities, such as the left atrial appendage.

Because the left atrial appendage is a long, tubular, hooked structure, safely deploying the cardiac device within the left atrium appendage requires not only careful precision, but also requires orienting the device perpendicular to the left appendage plane in order to ensure implant success of the cardiac device. Incorrectly positioning and deploying the cardiac device within the left atrial appendage may lead to ineffective treatment and increased likelihood of future heart complications, such as device embolism or Thrombus formation. Similar deployment precision issues are also present in the positioning and deploying of stents into the vascular system of patients. Incorrect positioning of the sheath, deployment wires and engaged catheters during such procedures can significantly impact the outcome.

### BRIEF SUMMARY OF THE INVENTION

Embodiments disclosed herein are directed toward a cardiac device deployment system that enables manipulation and control of the cardiac device during deployment into a target position in the vasculature, while reducing the risk of damaging proximal anatomy. For example, some embodiments provide a multi-lumen catheter with a dual-lumen sheath configured to receive a shapeable guide-wire through a first lumen and a cardiac device, deployed with a cardiac device delivery system, through a second lumen. The shapeable guidewire may be used in concert with the cardiac device delivery system, to manipulate the cardiac device relative to the target anatomy such as by bending and repositioning the distal end of the multi-lumen sheath.

For example, the cardiac device may be a WATCHMAN device, and the cardiac device delivery system may be a catheter shaped to fit within the second lumen, and designed to hold the cardiac device at a distal end. The shapeable guidewire may be shaped with a substantially smaller cross-sectional circumference, such that the first lumen may also have a substantially smaller cross-sectional circumference than the second lumen. The shapeable guide-wire may comprise a shape-memory material, such that the guide-wire may be manipulated into a predetermined shape configuration before being advanced within the first lumen and may be manipulated once advanced through the first lumen to align and reposition the distal end of the sheath. The target anatomy may include any bodily structure requiring a treatment with a device delivered by the multi-lumen sheath or catheter, such as the heart, lung, kidney, bladder, abdominal cavities, and the like. Within the heart, the target anatomy may include any fold, cavity, or appendage, including blood supply arteries and the left atrial appendage.

In some embodiments, a balloon may be used in conjunction with the guidewire to protect the proximate anatomy from accidental scraping or puncture damage. For example, the balloon may be deployed through one of the lumens in the sheath or multi-lumen catheter in order to provide a protective bumper between the cardiac walls and the shapeable guidewire. Alternatively and preferred, the balloon can be employed as an anchor to substantially fix the distal end of the sheath bearing the delivery catheters and wires, so that the surgeon can concentrate on positioning the implant, knowing that the distal end of the sheath will substantially maintain its anchored position anchored by the balloon and balloon wire.

In one embodiment of the disclosure, a multi-lumen catheter device includes a sheath with a first lumen and a second lumen, each disposed within the sheath. The second lumen may have a cross-sectional circumference that is greater than the cross sectional circumference of the first lumen. For example, the first lumen may be a guidewire lumen shaped to receive a shapeable guidewire, and the second lumen may be a device lumen shaped to allow the cardiac device to move longitudinally from the proximal end of the catheter to the distal end of the catheter. The shapeable guidewire may be substantially smaller in diameter than the cardiac device and may incorporate a malleable material with shape memory. Due to the shape memory material, the distal end of the guide-wire may be articulated into a first shape prior to insertion into the second lumen, may bend into a second shape during deployment through the second lumen, and may reflex in to a third shape that is substantially similar to the first shape after the distal end of the guidewire extends beyond the distal end of the sheath.

In another embodiment, a multi-lumen catheter device includes a sheath with a first lumen, a second lumen, and a third lumen disposed within the sheath. The second lumen may have a cross sectional circumference greater than the cross sectional circumference of the first lumen and the cross sectional circumference of the third lumen. For example, the first lumen may be a guide-wire lumen shaped to receive a shapeable guide-wire, the second lumen may be device lumen shaped to allow the cardiac device to move longitudinally from the proximal end of the catheter to the distal end of the catheter engaged within the multi lumen sheath. The third lumen may be a balloon lumen configured to receive and translate a balloon deployment system therethrough. The balloon deployment system may include a balloon located at the distal end of a balloon wire or guidewire.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein, which are defined solely by the claims attached hereto.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a diagram illustrating a cross-sectional view of a multi-lumen catheter, consistent with some embodiments disclosed herein.

FIG. 2 is a diagram illustrating a cross-sectional view of the multi-lumen catheter with a shapeable guidewire inserted, consistent with some embodiments disclosed herein.

FIG. 3 is a diagram illustrating a multi-lumen catheter deployed into the left atrium with a cardiac device positioned to seal the left atrial appendage, consistent with embodiments disclosed herein.

FIG. 4 is a diagram illustrating a multi-lumen catheter with a balloon guidewire and cardiac device disposed therein, consistent with some embodiments disclosed herein.

FIG. 5 is a diagram illustrating a cross-sectional view of a multi-lumen catheter, consistent with some embodiments disclosed herein.

FIG. 6 is a diagram illustrating a cross-sectional view of a multi-lumen catheter with a shapeable guidewire, a cardiac device, and a balloon guidewire inserted, consistent with some embodiments disclosed herein.

FIG. 7 is a diagram illustrating a multi-lumen catheter deployed into the left atrium with a cardiac device positioned to seal the left atrial appendage, consistent with some embodiments disclosed herein.

FIG. 8 is a flow chart illustrating a method for deploying a multi-lumen catheter into a target anatomy, consistent with some embodiments of this disclosure.

FIG. 9 is a flow chart illustrating a method for inserting a guidewire into a multi-lumen catheter, consistent with some embodiments disclosed herein.

FIG. 10 is a flow chart illustrating a method for manipulating a cardiac device with a shapeable guidewire within an atrium target anatomy, consistent with some embodiments disclosed herein.

FIG. 11 is a flow chart illustrating a method for inserting a distal balloon guidewire end into a multi-lumen catheter consistent with some embodiments of this disclosure.

FIG. 12 is a flow chart illustrating a method for deploying a multi-lumen catheter into an atrium target anatomy, consistent with some embodiments disclosed herein.

FIG. 13 depicts a particularly preferred mode of the MULTI-LUMEN delivery device herein, showing a sheath having lumens for a shapeable guidewire, a cardiac device and guidewire therefor, and a balloon with a balloon wire communicating therethrough, consistent with some embodiments disclosed herein.

FIG. 14 shows another particularly preferred mode of the multi-lumen delivery device herein, showing a sheath including lumens for a shapeable guidewire, a cardiac device and guidewire therefore, and a balloon engaged at a distal end of a balloon wire, all communicating through respective lumens from a proximal to distal end of the sheath.

FIG. 15 is a flow chart illustrating a method for deploying the multi-lumen catheter to position the distal end of the sheath in an anchored position adjacent the target anatomy for the cardiac or implant device.

The figures are not intended to be exhaustive or to limit the invention to the precise form of the device and method disclosed herein. It should be understood that the invention can be practiced with modification and alteration, and that the disclosed technology shall be limited only by the claims and the equivalents thereof as would occur to those skilled in the art.

### DETAILED DESCRIPTION OF VARIOUS EMBODIMENTS

The following description is not to be taken in a limiting sense, but is made merely for the purpose of describing the general principles of the disclosed embodiments. The present embodiments address the problems described in the background while also addressing other additional problems as will be seen from the following detailed description. Numerous specific details are set forth to provide a full understanding of various aspects of the subject disclosure. It will be apparent, however, to one ordinarily skilled in the art, that various aspects of the subject disclosure may be practiced without some of these specific details. In other instances, well-known structures and techniques have not been shown in detail to avoid unnecessarily obscuring the subject disclosure.

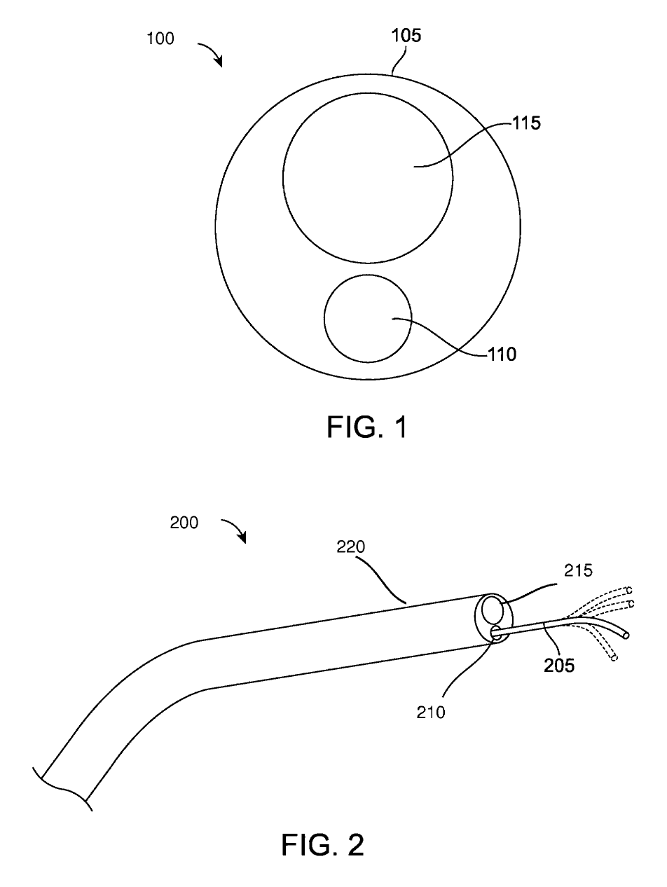

As illustrated in FIG. 1, one embodiment of the disclosure is a multi-lumen catheter device 100 that may include sheath 105 with a first lumen 110 and a second lumen 115 disposed within sheath 105. The first lumen 110 may have a cross-sectional circumference smaller than the cross-sectional circumference of the second lumen 115, and the sum of the cross-sectional diameters of first lumen 110 and second lumen 115 is less than the cross-sectional diameter of the sheath 105. In some embodiments, first lumen 110 ranges between 3 and 10 French. In some embodiments, the second lumen 115 range between 10 and 30 French. Lumens of different sizes may be selected according to the applicable constraints, such as the requirement that the shapeable guidewire fit within the first lumen, the cardiac device fit inside the second lumen, both lumens to fit within the sheath, and the sheath to fit within all of the vasculature in the approach from a catheter entry site to the target anatomy (e.g., the vasculature between a femoral entry point and the heart).

In some embodiments, the multi-lumen catheter must be sized for use on a smaller patient anatomy (e.g., pediatric patients or animals), such that the multi-lumen catheter must be small enough to be inserted through a smaller anatomy, while also large enough to insert the proper device or tools through the multi-lumen catheter. In some examples, first lumen 110 may range between 5 and 8 French and the second lumen 115 may range between 10 and 14 French.

In another example, the multi-lumen catheter 100 may be utilized for veterinary treatment for an animal, such as a dog, cat, horse, cow, pig, and the like. Should the multi-lumen catheter be used to treat a horse for example, then the sheath and enclosed lumens should be sized to fit within the vasculature of a horse. For example, the first lumen, 110 may range between 10 and 15 French and the second lumen 115 may range between 15 and 25 French to accommodate the larger vascular anatomy of a horse.

As illustrated in FIG. 2, the first lumen may be a guidewire lumen 210 shaped to receive a shapeable guidewire 205 and the second lumen may be a device lumen 215 shaped to accept and enable the cardiac device to be advanced through the longitudinal axis of the device lumen using a device delivery catheter, starting at a proximal opening of the device lumen (not shown) and extending past a distal opening of the sheath 220. The shapeable guidewire 205 may be fabricated from a malleable material with shape memory enabling the shapeable guidewire 205 to be formed into a desired first shape configuration, held temporarily in a second shape configuration (e.g., as the guidewire moves through the guidewire lumen), and then released such that the malleable material reflexes into a third shape configuration that nearly approximates the first shape-configuration. For example, the malleable material may include aluminum, copper, silicone, stainless steel, titanium, tungsten, or other metals or composite materials. These materials may be fabricated into a shape-memory alloy (SMA) such as Fe—Mn—Si, Cu—Zn—Al, Cu—Al—Ni, or NiTi (Nitinol). One of ordinary skill in the art would recognize that other shape-memory materials may be used to fabricate the guidewire.

Referring back FIG. 2, the varied broken lines of the shapeable guidewire 205 illustrate, by way of example, the flexible bending and varied configurations the shapeable guidewire 205 may be capable of configuring into. The different shape configurations are illustrated for exemplary purposes. One of ordinary skill in the art would recognize that other shape configurations are possible and may be desired depending on the particular target anatomy.

As described, the distal end of the shapeable guidewire 205 may be articulated into a first shape prior to insertion into the guidewire lumen 210, bent to a second shape during deployment through the guidewire lumen 210, and may reflex in to a third shape that approximates the first shape after the distal end of the shapeable guidewire 205 extends beyond past the distal end of the sheath 220. In some examples, the guidewire may not fully reflex into a shape that approximates the third shape, such that the third shape may fall somewhere between the first shape and the second shape. In such a case, the first shape may be intentionally over-flexed with comparison to the desired third shape. For example, the guidewire may be initially bent further than the desired shape to compensate for the effect of running the guidewire through the guidewire lumen in an approach that may effectively straighten the guidewire, such that the guidewire does not completely reflex to its original shape configuration.

Once deployed through the guidewire lumen, the distal end of the guidewire may be used as a second point of contact on a proximal side of the cardiac device to enable precise manipulation of the cardiac device. In one example, the configured first shape of the shapeable guidewire 205 may be determinant upon the size and shape of the cardiac device to be deployed to the target anatomy, such as the left atrial appendage. In another example, the first configured shape of the distal end of the shapeable guidewire 205 may be determinant upon the shape and dimensions of the target anatomy, as well as the areas proximate to the target anatomy.

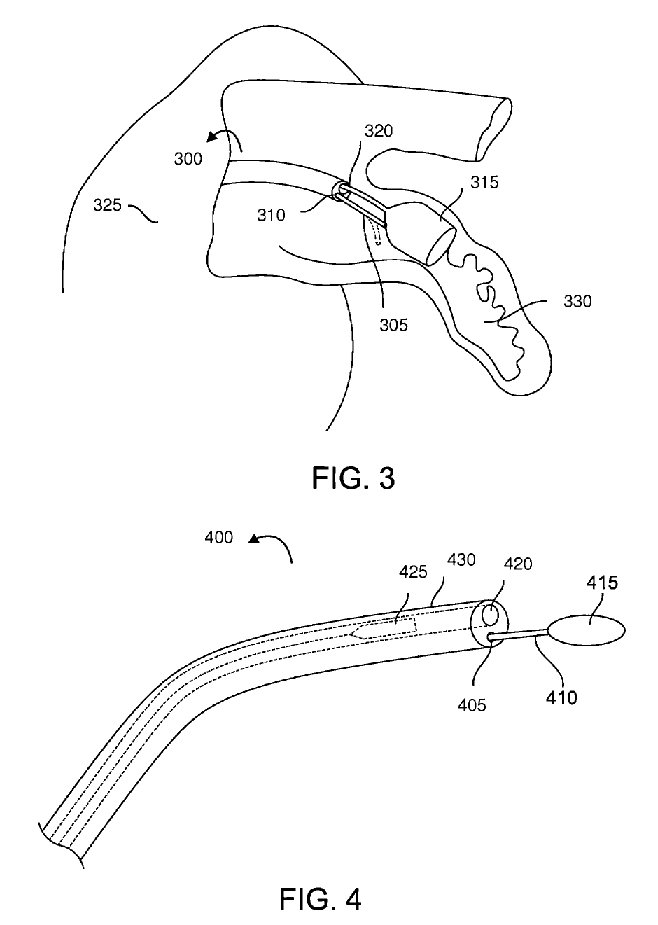

As illustrated in FIG. 3, a multi-lumen catheter 300 with a guidewire lumen 310 and a device lumen 320 allows for the simultaneous insertion, transport, and placement of shapeable guidewire 305, through a first lumen, and cardiac device 315, through a second lumen, from an entry point to a target anatomy. For example, the entry point may be an area on a subject's skin where the multi-lumen catheter may enter the vascular anatomy. Example entry points include a jugular vein, subclavian artery, subclavian vein, brachial artery, femoral arteries, and the femoral vein. In one embodiment, a shapeable guidewire 305 may be inserted through the guidewire lumen 310 and a cardiac device 315 may be inserted through the device lumen 320 to deploy the shapeable guidewire 305 and cardiac device 315 in to the left atrial appendage 330.

As further illustrated in FIG. 3, multi-lumen catheter 300 enables distal ends of the shapeable guidewire 305 and the cardiac device 315 to be simultaneously present within the left atrium 325. As such, the distal end of the shapeable guidewire 305 is used to orient and manipulate the deployment of the cardiac device 315 within the left atrial appendage 330 by applying more or less pressure to a proximal side of the cardiac device 315, in coordination with pressure applied to the proximal side of the cardiac device from its own deployment catheter, which is advanced through the device lumen of the multi-lumen catheter. Thus, coordinated pressure may be applied to each contact point through the guidewire and/or the deployment catheter as needed to effectively manipulate the cardiac device into its final position (e.g., to seal the left atrial appendage).

In other embodiments, as illustrated in FIG. 4, a multi-lumen catheter 400 may be configured to receive a balloon. For example, guidewire lumen 405 may be configured to receive a balloon deployment device 410 in addition to a shapeable guidewire (not shown), and device lumen 420 may be configured to receive a cardiac device 425. The balloon deployment device 410 may be a catheter, guidewire, or other balloon deployment device known in the art. Balloon deployment device 410 may include at its distal end balloon 415. In some embodiments, balloon 415 may be detachable from balloon deployment device 410. For example, balloon 415 may be detached from balloon deployment device 410 after the balloon is placed near the left atrial appendage. The balloon deployment device may then retracted, freeing the guidewire lumen 405 for use with a shapeable guidewire. The shapeable guidewire may then be advanced through guidewire lumen until the distal end of the shapeable guidewire extends beyond the distal end of sheath 430, but abuts against balloon 415, such that balloon 415 protects any internal anatomy from damage caused by moving the distal end of the shapeable guidewire within the target anatomy.

In other embodiments, balloon 415 may be affixed to the distal end of the balloon deployment device 410. For example, balloon 415, as affixed to the distal end of balloon deployment device 410, may be advanced through guidewire lumen 405 and pushed past the distal end of the sheath 430, and balloon 415 may be manipulated toward the left atrial appendage with balloon deployment device 410.

In several embodiments, either or both of the distal ends of the shapeable guidewire and balloon deployment device 410 include a radiopaque material, such that they will be visible using an x-ray imaging system. For example, the tip of the shapeable guidewire may incorporate a radiopaque material.

In some embodiments, balloon 415 at the distal end of balloon deployment device 410 may be configured in a deflated state prior to insertion into guidewire lumen 405, and the balloon may then be inflated after the balloon extends past the distal end of sheath 430. By way of example only, the inflated balloon 415 provides a protective bumper relative to its immediate vicinity, such as the vasculature, cardiac wall, or other proximate anatomy of the target anatomy. The protective bumper may protect the proximate anatomy from accidental scraping or puncture caused by the tools or devices deployed into the target anatomy using multi-lumen catheter 400. For example, deployed balloon 415 may be positioned between the atrium walls and the cardiac device 425 and/or shapeable guidewire (not shown), such that balloon 415 protects the atrium walls from being scratched or punctured from the shapeable guidewire.

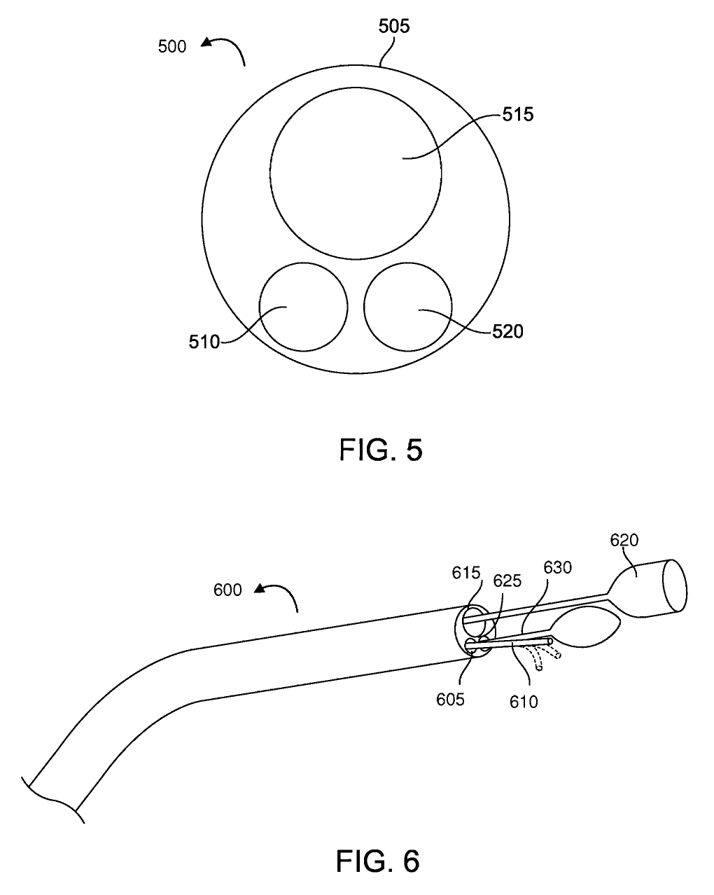

In other embodiments, as illustrated in FIG. 5, multi-lumen catheter device 500 may include three lumens. For example, a sheath 505 may include a first lumen 510, a second lumen 515, and a third lumen 520, each disposed within the sheath 505. The second lumen 515 is shaped to have a cross-sectional circumference greater than the cross-sectional circumference of the first lumen 510 and the second lumen 515, and the first lumen 510, second lumen 515, and third lumen 520 each fit within the cross-sectional circumference of the sheath. The determination of the select third lumen size may be determined upon the type of tool to be inserted through the third lumen. For example, the third lumen 520 may be between 5 and 20 French, or may be smaller or larger depending on the shape and size of the device being inserted.

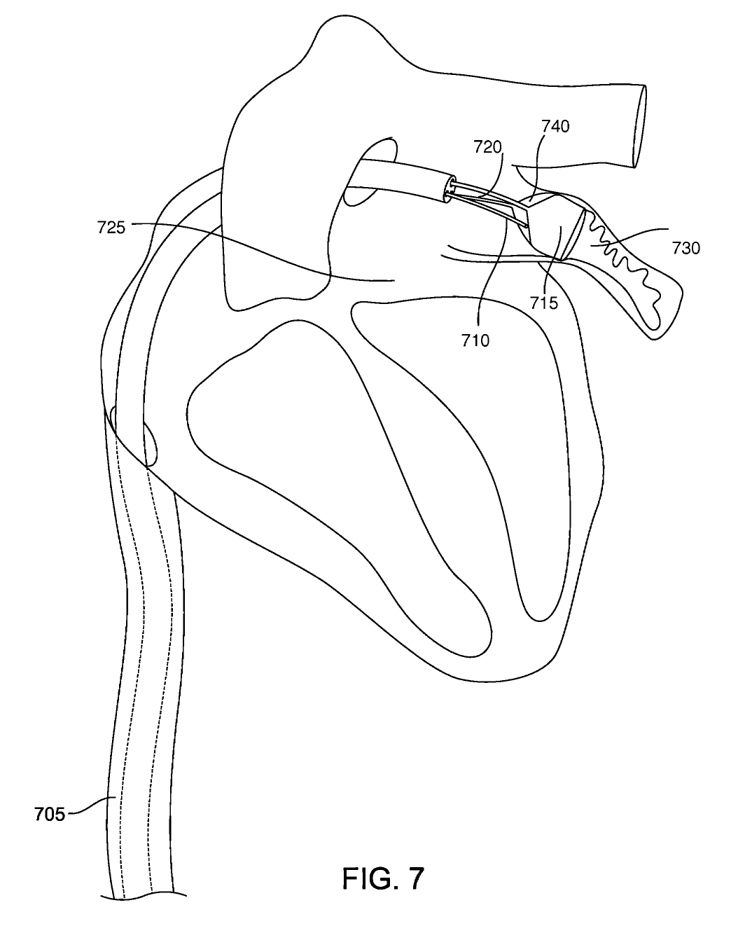

As illustrated in FIG. 6, the first lumen may be a guidewire lumen 605 configured to receive a shapeable guidewire 610, the second lumen may be a device lumen 615 configured to receive a cardiac device 620, and the third lumen may be a balloon lumen 625 configured to receive a balloon deployment device 630. In one embodiment, multi-lumen catheter 600 allows for the simultaneous insertion of shapeable guidewire 610, cardiac device 620, and balloon deployment device 630 from an entry point, such as a femoral artery 705, as further illustrated in FIG. 7. For example, with shapeable guidewire 710, cardiac device 715, and balloon deployment device 720 all simultaneously present in left atrium 725 near the left atrial appendage 730, shapeable guidewire 710 may guide and orient cardiac device 715 within the left atrial appendage 730, while balloon 740 provides a protective bumper to protect the atrial walls from the shapeable guidewire 710.

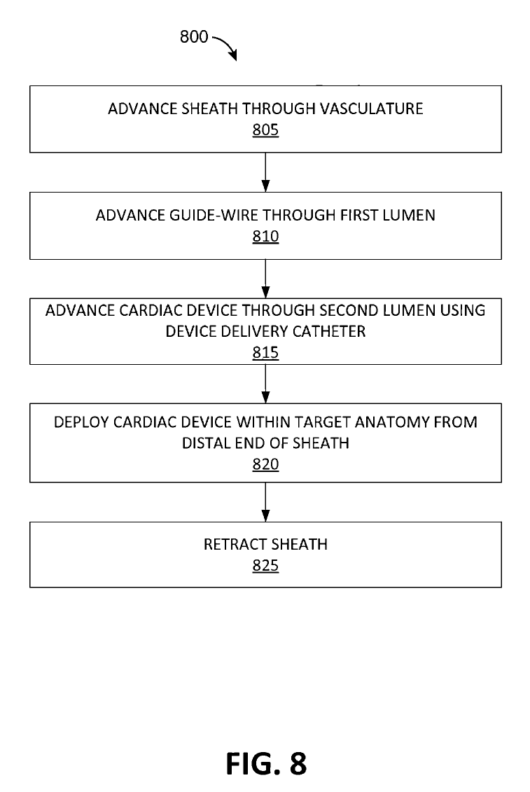

FIG. 8 is an example flow diagram that illustrates a method for deploying a multi-lumen catheter to deliver a shapeable guidewire and device to a designated target anatomy. As illustrated in FIG. 8, embodiments of method 800 include inserting a sheath end into an entry point at step 805. The insertion point may be the jugular vein, subclavian artery, subclavian vein, brachial artery, femoral arteries, the femoral vein, or any other entry point as known in the art.

Still referring to FIG. 8, the method may also include inserting the shapeable guidewire into the guidewire lumen, such that the distal end of the shapeable guidewire extends past the distal end of the sheath at step 810. The method may also include inserting a cardiac device through the device lumen and into the left atrium at step 815. The cardiac device may be positioned near the target anatomy, such as the left atrial appendage, and manipulated to mechanically align the cardiac device perpendicular to the left atrial appendage plane at step 820. As the cardiac device is deployed, the distal end of the sheath and shapeable guidewire may be retracted at step 825.

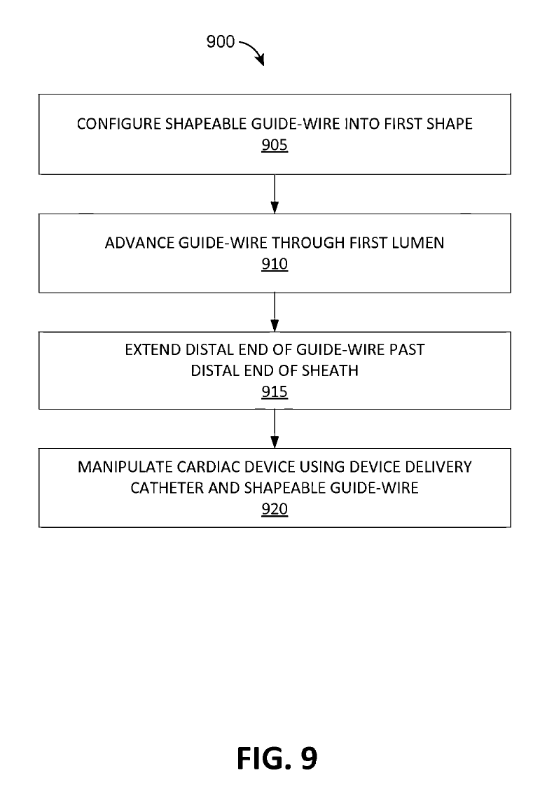

FIG. 9 is an example flow diagram that illustrates a method 900 for preparing and inserting the shapeable guidewire into the guidewire lumen. Method 900 may include configuring the shapeable guidewire into a first shape at step 905. By way of example, the configuration of the first shape may be determinant upon the size and shape of the selected device to be deployed and anticipated approach to the target anatomy. For example, if the approach to the target anatomy requires that the cardiac device take a downward slope after leaving the distal end of the sheath, to reach the target anatomy, then the shapeable guidewire may be bent at a distal end to approximate the same downward bend. In some examples, the shapeable guidewire must be initially bent more than the approach to the target anatomy would require, because the travel through the guidewire lumen will partially re-straighten the guidewire. Even though the guidewire may comprise a shape-memory material, once the distal end of the guidewire extends beyond the distal end of the sheath, the guidewire may not completely regain its initial shape, but instead may enter into a third shape that closely approximates the initial shape. Accordingly, slightly over-bending the guidewire into the first shape may compensate for the straightening effect that occurs during transport through the guidewire lumen.

The guidewire must be sufficiently large with respect to its cross-sectional diameter to maintain its shape and sufficient tensile strength to push, manipulate, and/or orient the cardiac device within the target anatomy, but also must be sufficiently small with respect to its cross-sectional diameter to fit within the sheath, and ultimately, the vasculature, alongside the cardiac device delivery system and lumen.

In one example implementation of the disclosure, method 900 includes disposing the shapeable guidewire through the guidewire lumen at step 910. As described above, because the shapeable guidewire is transported through the restrictive confinement of the shapeable guidewire lumen, the configured first shape of the distal guidewire end may transform into a second shape (e.g., the shapeable guidewire may straighten during transport through the guidewire lumen). The shapeable guidewire may reflex in to a third shape that is substantially similar to the first shape after shapeable guidewire is extended past the confinement of the distal end of the sheath. As the distal end of the shapeable guidewire reaches the left atrium, the shapeable guidewire, in concert with the cardiac device delivery system, manipulates, orients, aligns, and guides the cardiac device within the left atrial appendage at step 920.

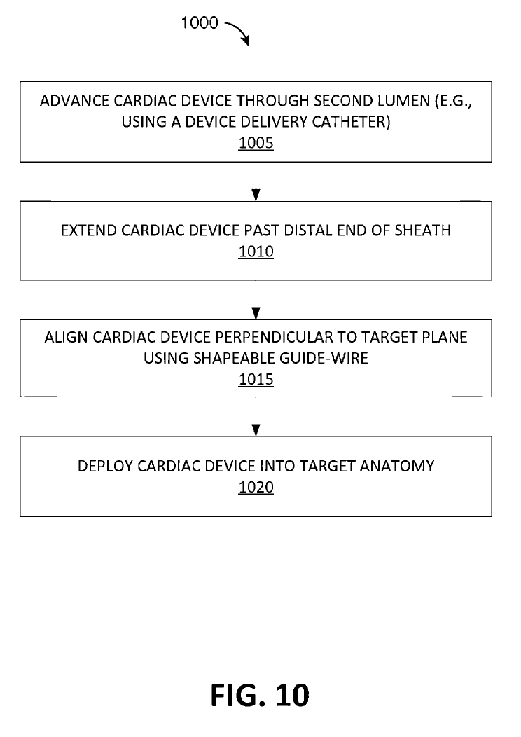

FIG. 10 is a flow diagram that illustrates method 1000 for preparing and deploying a cardiac device with a multi-lumen catheter. As shown, a method for preparing and deploying a cardiac device with a multi-lumen catheter includes disposing the cardiac device through a device lumen. The method may also include extending the cardiac device past the distal end and in proximity to the target anatomy (e.g., into the left atrium) at step 1010. The method may also include aligning the cardiac device to a plane perpendicular to a target plane (e.g., the desired radial plane for the cardiac device, wherein the radial plane is orthogonal to the surrounding target anatomy walls), at step 1015. The method may also include deploying the cardiac device at step 1020. For example, the cardiac device may be opened into a fully deployed position with an enlarged cross-sectional diameter matching the cross-sectional diameter of the target anatomy, and the sheath may be retracted from the cardiac device, leaving the cardiac device in place.

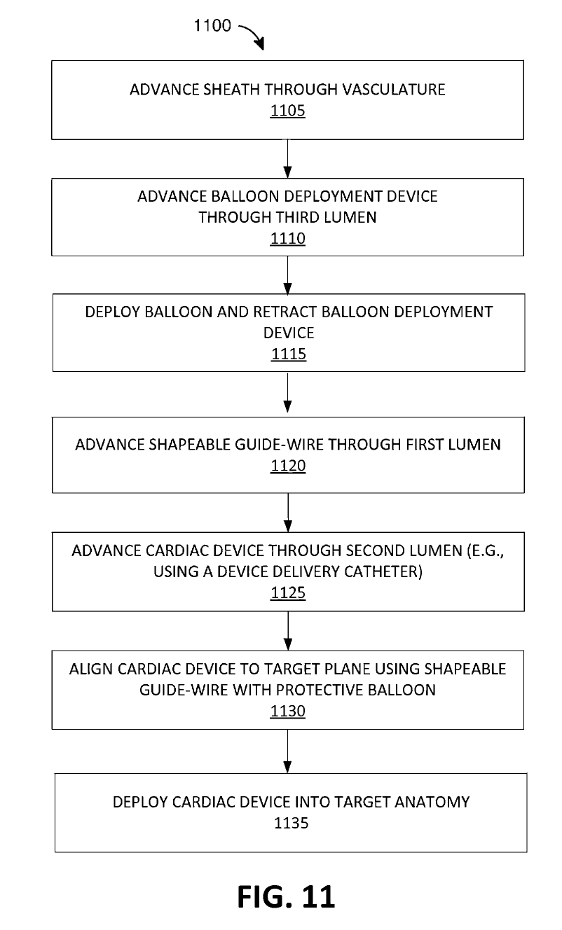

FIG. 11 is a flow diagram that illustrates a method 1100 for protecting the proximate areas of the target anatomy. The method includes disposing a sheath through the vasculature to reach a target anatomy at step 1105. The method may also include disposing a balloon delivery device through the guidewire lumen, such that the distal end of the balloon delivery device extends past the distal end of the sheath at step 1110. For example, the balloon delivery device may be a balloon guidewire.

In one example, the balloon attached at the distal end of the balloon delivery device is transported through the guidewire lumen in a deflated state. The balloon is then inflated after the balloon extends past the distal end of the sheath and in close proximity to the target anatomy. In one embodiment, the balloon is placed near the target anatomy (e.g., the left atrial appendage), the balloon is detached from the distal end of the balloon delivery device, and the balloon delivery device is retraced from the multi-lumen catheter and entry point at step 1115.

In some embodiments, the method may also include disposing a shapeable guidewire through the guidewire lumen at step 1120. The shapeable guidewire may be manipulated to abut against the balloon, such that the balloon provides a protective bumper between the target anatomy and the distal end of the shapeable guidewire. The method may also include disposing a cardiac device through the device lumen at step 1125.

In further embodiments, the method may also include using the shapeable guidewire and a cardiac device delivery system (e.g., a guidewire designed to deploy the cardiac device through the device lumen) in concert to align the cardiac device to a target plane at step 1130. During the alignment process, the balloon continues to protect the surrounding anatomy from accidental scraping or puncture damage from the shapeable guidewire. The cardiac device may then be deployed into the target anatomy at step 1135.

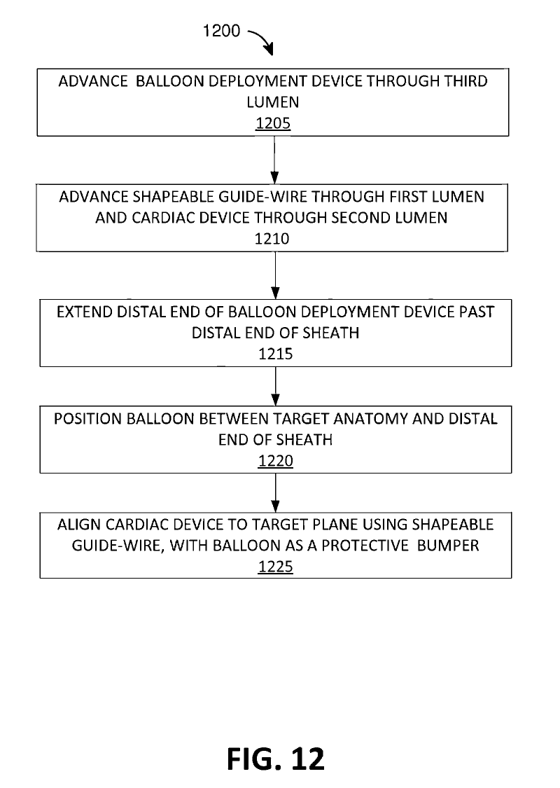

FIG. 12 is a flow diagram that illustrates a method 1200 for deploying a shapeable guidewire, cardiac device, and balloon guidewire through a multi-lumen catheter to a designated target anatomy. Method 1200 provides an example of maneuvering a cardiac device into the target anatomy while reducing the risk of damaging the proximate anatomy. The method includes disposing a balloon deployment device through a third lumen at step 1205. For example, the balloon deployment device may be advanced through the third lumen, the shapeable guidewire may be advanced through the first lumen, and the cardiac device may be advanced through the second lumen using a device delivery catheter, all at the same time, at step 1215. The method may also include extending the distal end of the balloon deployment device (e.g., a balloon guidewire) past the distal end of the sheath at step 1215.

The shapeable guidewire may be advanced through the guidewire lumen such that the distal end of the shapeable guidewire extends past the distal end of the sheath, and positioned to abut with a proximal end of the balloon at step 1220, such that the balloon is positioned between the shapeable guidewire and the target anatomy. By way of example, the shapeable guidewire and the balloon guidewire, located at the distal end of the multi lumen sheath, may then be simultaneously manipulated towards the target anatomy. In another example, prior to inserting the shapeable guidewire into the guidewire lumen, the distal end of the shapeable guidewire end may be configured to a first shape, as described with respect to FIG. 9.

The cardiac device may be advanced through the cardiac lumen using a device delivery catheter, and advanced towards the target anatomy. In one example, with the balloon guidewire and shapeable guidewire already present within the target anatomy, the cardiac device may be located in close proximity to the target anatomy such that the shapeable guidewire can align, manipulate, and guide the placement of the cardiac device in a target plane (e.g., perpendicular to a longitudinal axis of the left atrial appendage) at step 1225. The cardiac device may then be deployed and the sheath retracted.

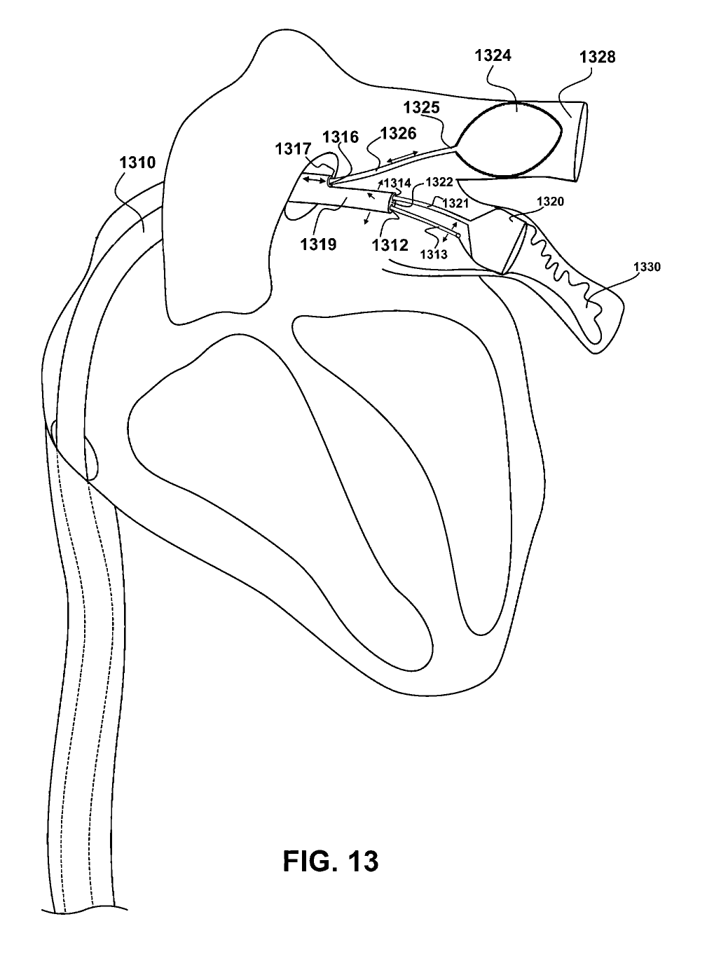

Depicted in FIG. 13, is a particularly preferred mode of the multi-lumen delivery device herein. As shown in FIG. 13, the multi-lumen sheath 1310 has a first lumen 1312 adapted for translation of the shapeable guidewire 1313 therethrough. The multi-lumen sheath 1310 also has the second lumen 1314 adapted for translation of the cardiac device 1320 therethrough as well as a third lumen 1316 which terminates a distance from the distal end 1322 of the multi lumen sheath 1310.

This mode of the device and method herein, is particularly preferred as it provides a means for the surgeon to achieve an anchor for the multi-lumen sheath 1310 such that the distal end 1322 is held in position once a balloon 1324 engaged to a balloon wire 1326 of the balloon deployment device 1325 is inflated and anchored at an anchoring position in an intersecting or adjacent blood vessel such as the pulmonary vein 1328. Once anchored, the balloon wire 1326 engaged with the balloon 1324 forms a fixed rail on which the sheath 1310 can be translated toward and away from the target anatomy.

In this mode of the device herein, the multi-lumen sheath 1310 can be advanced to position the distal end 1322 adjacent the target anatomy such as the atrial appendage 1330, wherein the balloon deployment device 1325 is translated through the third lumen 1316 whereupon it exits the third lumen 1316 and the multi-lumen sheath 1310 a distance from the distal end 1322. This is important because it allows the distal end 1322 to be manipulated for lateral position on the balloon wire 1326 and extension portion 1319 to be manipulated for angle and axis by the shapeable guidewire 1313, after the balloon 1324 is inflated to anchor it. As shown the balloon 1324 is inflated to anchor it in the pulmonary vein 1328. So anchored, the engaged balloon wire 1324 is also fixed in position anchored to the balloon 1324. This allows the user to translate the multi-lumen sheath 1310 toward and away from the anchored balloon 1324 and manipulate the position of the distal end 1322 within the blood vessel. A lock, such as a clamp (now shown), can engage the balloon wire 1326 to the multi-lumen sheath 1310 at the proximal end, thereby fixing the position of the multi-lumen sheath on the balloon wire 1326 and fixing the position of the distal end 1322.

With the balloon 1324 anchored, and the lateral position of the distal end 1322 substantially fixed by the lock or clamp holding the multi lumen sheath 1310 on the balloon wire 1326, the surgeon can then use the shapeable guidewire 1313 to bend and manipulate an angle and axial position or alignment of an extension portion 1319 of the multi-lumen sheath 1310 within the vein or artery. The sheath 1310 is flexible so manipulating the shapeable guidewire 1313 within or projecting from the extension portion 1319 allows for easy adjusting of the angle of the extension portion 1319 which extends between the exit aperture 1317 of the third lumen 1316 and the distal end 1322 of the multi lumen sheath 1310. This allows the user to position the distal end 1322 and the axis of the lumen carrying the implant correctly.

Additionally, with the lateral position of the distal end 1322 fixed by the engaged balloon wire 1326 and anchored balloon 1324, it makes it much easier for the surgeon to employ the shapable guidewire 1313 to place the distal end 1322 and axis of the second lumen 1314 or a lumen carrying the device to be implanted, aligned with the axis of the atrial appendage 1330 or other target anatomy for implantation of a cardiac device 1320 such as a stent or the WORKMAN or another device where a precise placement prior to final implantation is extremely important. Further, should positioning of the distal end 1322 laterally be required, the sheath can be slid in its axial engagement on the balloon wire 1326.

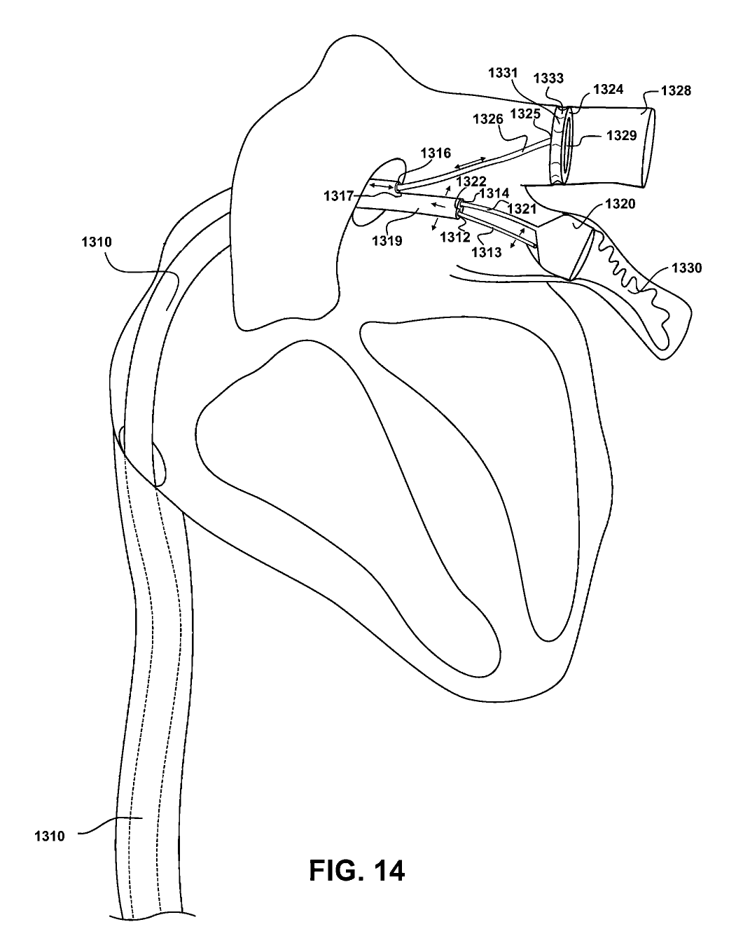

The device shown in FIG. 14, operates in a substantially similar fashion to the device as shown in FIG. 13. However, the balloon 1324 in this mode when anchoring in a blood vessel intersecting or adjacent the target anatomy for the cardiac device 1320 or other implant, includes an opening 1329 therein to allow for the passage of blood flow once the balloon 1324 is anchored in the blood vessel or body tissue of choice. The balloon 1324 in this mode has an appearance somewhat like a donut, and once inflated, a perimeter edge 1331 contacts and compresses against the interior of the chosen blood vessel or body tissue, shown as the pulmonary vein 1328 for convenience. Additionally, an annular recess 1333 can depend into the surface of the perimeter edge 1331 of the balloon 1324. This annular recess 1333 in the inflated balloon 1324 causes tissue surrounding the balloon 1324 against which the perimeter 1331 compressively engages, to protrude and engage slightly into the annular recess 1333. This engagement of tissue into the annular recess 1333 significantly enhances the anchoring of the balloon 1324 into the chosen blood vessel or body passage or the like.

The annular recess 1333 could also be formed into other shaped balloons such as the balloon 1324 of FIG. 13.

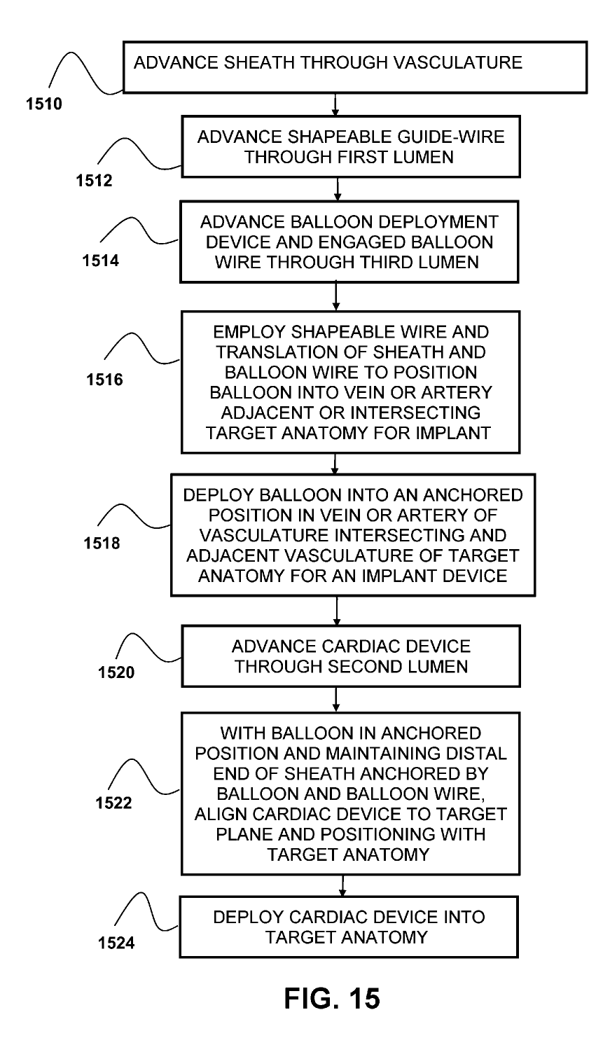

FIG. 15 is a flow chart illustrating a method for deploying the multi-lumen sheath 1310 or catheter device of FIGS. 13 and 14, to position the distal end adjacent the target anatomy for the cardiac or implant device, and then anchor it in place using the inflated balloon and a locked connection of the balloon wire to the multi-lumen sheath. Any numerals referring to components are references to those in FIGS. 13-14.

As shown in a first step 1510, and using the device herein such as in FIGS. 13 and 14, the sheath is advanced through the vasculature of the patient, to position the distal end proximal to the desired target anatomy for an implant such as a cardiac device. In a subsequent step 1512 to the first step 1510, the shapeable guidewire is advanced through a first lumen in the multi lumen sheath or catheter. In a subsequent step 1514 to the first step 1510, a balloon deployment device with a balloon engaged to a balloon wire, is translated through the third lumen to an exit aperture 1317. Subsequent to step 1514, the balloon 1324 at the end of the balloon wire 1326, is deployed 1518 by being inflated and anchored in position in a chosen vein or artery or body cavity proximate to the target anatomy but preferably in a vascular passage intersecting or adjacent that of the target anatomy. The anchoring of the balloon 1324 secures the engaged balloon wire 1326 in place allowing the user, such as a surgeon, to thereafter translate the multi-lumen sheath 1310 or catheter on the balloon wire 1326 and thereby reposition the distal end 1322 with accuracy. As noted, a lock or clamp can be engaged between the balloon wire 1326 and the multi-lumen sheath 1310 at any time, to fix the sheath in position so that the surgeon can concentrate on moving the distal end 1322 to the correct angle and axial position.

In another step 1520 the cardiac device 1320 is advanced through the second lumen 1324 and to an exit therefrom at the distal end 1322. To align the cardiac device 1522 with the target anatomy plane, the distal end of the multi-lumen sheath 1310 or catheter can be adjusted in angle and lateral position on the balloon wire 1326, to a proper position which will axially center the cardiac device 1320 and place in the proper plane or laterally advanced position. This aligning of the cardiac device 1522 can be accomplished by the surgeon translating the entire multi-lumen sheath 1310 toward or away from the target anatomy with the balloon 1324 anchored and rending the balloon wire 1326 to form essentially a rail for such translation.

Additionally or in combination with the translation of the sheath, this aligning 1522 of the cardiac device can also be accomplished by manipulation of the shapeable wire 1313, which will cause the extension portion 1319 of the sheath, to change axial positioning since the fixed balloon wire 1326 holds the multi lumen sheath at and on the opposite side of the exit aperture 1317 in place. Essentially the extension portion 1319 can be tilted in any of four directions or axes, by the manipulation of the shapeable guidewire 1313.

In a final step, with the distal end 1322 of the multi-lumen sheath positioned at the correct angle and axial alignment by the previous alignment 1522, deployment 1524 of the device into the target anatomy occurs. Proper alignment 1522 of course can be ascertained during that step and prior to this step of deployment 1524 by conventional means such as ultrasound or fluoroscopy.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A cardiac device delivery system comprising:
a sheath extending from a first end, to a distal end, said sheath having a first lumen and a second lumen and a third lumen, disposed therein;
a shapeable guidewire having a cross-sectional profile shaped to fit within the first lumen;
said first lumen is shaped to have a first cross-sectional circumference;
said second lumen having a second cross-sectional circumference greater than the first cross-sectional circumference;
a shapeable guidewire with a cross-sectional profile shaped to fit within the first lumen;
a device delivery catheter with a cross-sectional profile shaped to fit within the second lumen;
a balloon deployment device shaped to fit within the third lumen having, said balloon deployment device having an inflatable balloon engaged to a distal end of a balloon wire; and
a cardiac device having an un-deployed cross-sectional circumference when the cardiac device is transported through the second lumen and abutted against a distal end of the device delivery catheter, and having a deployed cross-sectional circumference larger than the un-deployed cross-sectional circumference when deployed within a target anatomy.

2. The system of claim 1, additionally comprising:
said first lumen exiting said sheath at said distal end;
said second lumen exiting said sheath at said distal end;
said third lumen exiting said sheath at an exit aperture, said exit aperture positioned a distance from said distal end in-between said first end and said distal end;
an extension portion of said sheath extending between said exit aperture and said distal end of said sheath;
said extension portion being bendable by manipulation of said shapeable guidewire within said first lumen; and
wherein an angle of said extension portion and a portion of said second lumen therein, is adjustable by said manipulation of said shapeable guidewire to thereby axially position said cardiac device relative to said target anatomy.

3. The system of claim 2, additionally comprising:
an extension of said balloon wire from said exit aperture positioning said balloon to an anchor position; and
inflation of said balloon at said anchor position fixing said balloon wire in position and forming a rail for translation of said sheath thereon.

4. The system of claim 1, additionally comprising:
an opening communicating through said balloon from a first side thereof engaged with said balloon wire, to an opposite said from said first side.

5. The system of claim 2, additionally comprising:
an opening communicating through said balloon from a first side thereof engaged with said balloon wire, to an opposite said from said first side.

6. The system of claim 3, additionally comprising:
an opening communicating through said balloon from a first side thereof engaged with said balloon wire, to an opposite side from said first side.

7. The system of claim 1, wherein the shapeable guidewire comprises a shape-memory alloy.

8. The system of claim 7, wherein the shape-memory alloy comprises Fe—Mn—Si, Cu—Zn—Al, Cu—Al—Ni, or NiTi.

9. The system of claim 2, wherein the target anatomy is located within a human heart.

10. The system of claim 2, wherein an angle of said extension portion and a portion of said second lumen therein, is adjustable by said manipulation of said shapeable guidewire to thereby manipulate a plane of orientation of the cardiac device extending from said second lumen, to match a target plane within the target anatomy.

11. The system of claim 3, wherein an angle of said extension portion and a portion of said second lumen therein, is adjustable by said manipulation of said shapeable guidewire to thereby manipulate a plane of orientation of the cardiac device extending from said second lumen, to match a target plane within the target anatomy.

12. The system of claim 5, wherein an angle of said extension portion and a portion of said second lumen therein, is adjustable by said manipulation of said shapeable guidewire to thereby manipulate a plane of orientation of the cardiac device extending from said second lumen, to match a target plane within the target anatomy.

13. The system of claim 6, wherein an angle of said extension portion and a portion of said second lumen therein, is adjustable by said manipulation of said shapeable guidewire to thereby manipulate a plane of orientation of the cardiac device extending from said second lumen, to match a target plane within the target anatomy.

14. The system of claim 1, additionally comprising:
an annular recess depending into a perimeter of said balloon.

15. The system of claim 2, additionally comprising:
an annular recess depending into a perimeter of said balloon.

16. The system of claim 3, additionally comprising:
an annular recess depending into a perimeter of said balloon.

17. The system of claim 4, additionally comprising:
an annular recess depending into a perimeter of said balloon.

18. The system of claim 5, additionally comprising:
an annular recess depending into a perimeter of said balloon.

19. A method for deploying the device of claim 2, comprising the steps of:
advancing said sheath through the vasculature of a patient;
advancing said shapeable guidewire through said first lumen;
advancing said balloon deployment device through said third lumen;
advancing said balloon engaged to said balloon wire to extend to an anchoring position adjacent to said target anatomy;
inflating said balloon into said anchoring position;
advancing said cardiac device through said second lumen toward said target anatomy;
manipulating said shapeable guidewire within said first lumen to bend said extension portion to manipulate a plane of orientation of the cardiac device extending from said second lumen, to match a target plane within the target anatomy; and
deploying the cardiac device into the target anatomy.

20. The method of claim 19, wherein the target anatomy is located within a human heart.
